(19) United States
(12) Patent Application Publication
(10) Pub. No.: US 2025/0303554 A1
(43) Pub. Date: Oct. 2, 2025

(54) WIRE TENSION COMPENSATORY ADJUSTMENT DEVICE

(71) Applicant: ROEN Surgical, Inc., Daejeon (KR)

(72) Inventors: Un-Je YANG, Daejeon (KR); Dong-Ho LEE, Daejeon (KR); Duk-Yoo KONG, Daejeon (KR); Duk-Sang KIM, Daejeon (KR); Chan-Soon LIM, Daejeon (KR); Joon Hwan KIM, Daejeon (KR)

(73) Assignee: ROEN Surgical, Inc., Daejeon (KR)

(21) Appl. No.: 18/709,691

(22) PCT Filed: Nov. 15, 2022
(86) PCT No.: PCT/KR2022/017967

§ 371 Date: Oct. 10, 2024

(30) Foreign Application Priority Data
Nov. 15, 2021 (KR) 10-2021-0156232
Nov. 14, 2022 (KR) 10-2022-0151534

(51) Int. Cl. B25J 9/10; A61B 34/00

(52) U.S. Cl. CPC B25J 9/1045; A61B 34/71; A61B 2034/715

(57) ABSTRACT

A wire tension compensatory adjustment device according to one embodiment may comprise: driving wires, which comprise a first driving wire including one end thereof, and a second driving wire including the other end thereof, the first driving wire and the second driving wire being integrated; a first wire reel part capable of winding or unwinding the first driving wire; a second wire reel part capable of winding or unwinding the second driving wire; a shaft which is connected to the first wire reel part and the second wire reel part, and which rotates the first wire reel part and the second wire reel part; a motor, which applies power to the shaft so as to rotate the shaft; and a preload-adjusting part, which applies a preload to the driving wires so as to prevent drooping of the driving wires and maintain the tension of the driving wires.

### TECHNICAL FIELD

The present disclosure relates to a wire tension compensatory adjustment device. Specifically, the present disclosure relates to an adjustment device that is able to compensate for tension of a wire by applying a pre-load to the wire through an elastic body.

### BACKGROUND ART

A conventional wire tension adjustment device 10 includes a first driving fixed pulley part, and first and second tension adjustment fixed pulley parts 12a and 12b inside a body thereof to drive a wire as illustrated in FIG. 5. The first driving fixed pulley part 11 rotates in one direction in conjunction with the operation of a motor (not shown) when the motor is operated by a control part (not shown). In this case, when a high load is applied to a surgical instrument 93, a wire 92 may elongate. Depending on the state change of the wire 92, the wire may deviate from a pulley path or may not produce a desired traction force. As a result, the position control or directional control of the surgical instrument 93 may not be efficient.

### DISCLOSURE

### Technical Problem

The objective of one embodiment is to provide a device for maintaining the tension of a wire by compensating for deformation of the wire.

### Technical Solution

A wire tension compensatory adjustment device according to an embodiment includes: a driving wire, wherein the driving wire comprises a first driving wire comprising a first end thereof and a second driving wire comprising a second end thereof, with the first driving wire and the second driving wire being formed integrally; a first wire reel part capable of winding or unwinding the first driving wire; a second wire reel part capable of winding or unwinding the second driving wire; a shaft being connected to the first wire reel part and the second wire reel part to rotate the first wire reel part and the second wire reel part; and a preload-adjusting part configured to apply a preload to the driving wire to prevent slack of the driving wire and maintain tension of the driving wire.

In an embodiment, the preload-adjusting part may operate in a passive manner.

In an embodiment, the preload-adjusting part may include: a preload applying part configured to apply a preload to the driving wire by transmitting power to the first wire reel part or the second wire reel part; a preload base configured to support the preload applying part; and a preload cover configured to be connected to the preload base and to cover the preload applying part.

The preload applying part may include: an elastic body configured to apply a preload to the driving wire; and a rotating body configured to rotate the elastic body.

In an embodiment, the elastic body may be a spiral spring or a torsional spring.

In an embodiment, the rotating body may be a ratchet wheel or a one-way pulley.

In an embodiment, the preload-adjusting part may be provided with a plurality of preload-adjusting parts, wherein among the plurality of preload-adjusting parts, a first preload-adjusting part may be connected to the first wire reel part, and a second preload-adjusting part may be connected to the second wire reel part.

In an embodiment, the wire tension compensatory adjustment device may further include: a motor configured to apply power to the shaft to rotate the shaft.

In an embodiment, the motor may be provided with a plurality of motors, wherein among the motors, a first motor may drive the first wire reel part, and a second motor may drive the second wire reel part.

In an embodiment, the first preload-adjusting part may be disposed between the first wire reel part and the first motor, and the second preload-adjusting part may be disposed between the second wire reel part and the second motor.

In an embodiment, the first driving wire entering the first wire reel part may be wound in a first direction, and the second driving wire entering the second wire reel part may be wound in a second direction opposite to the first direction.

In an embodiment, when any one wire reel part of the first wire reel part and the second wire reel part has threads and grooves formed therein to wind the driving wire, one remaining wire reel part thereof may have threads and grooves formed therein to unwind the driving wire.

In an embodiment, the first wire reel part and the second wire reel part may rotate in the same directions when winding or unwinding the driving wire.

### Advantageous Effects

According to the present disclosure as described above, when a high load is applied to a surgical instrument or when a state change occurs in a wire due to long-term use, that is, when the wire is elongated, the elongation of the wire is compensated by applying a preload through elasticity, thereby facilitating smooth position control and directional control of the surgical instrument.

### DESCRIPTION OF DRAWINGS

The following drawings attached to this specification illustrate a preferred embodiment of the present disclosure, and serve to further aid the understanding of the technical idea of the present disclosure along with a detailed description of the present disclosure, so the present disclosure should not be construed as limited to matters described in the drawings.

FIG. 1 is a diagram schematically illustrating a wire tension compensatory adjustment device according to an embodiment, wherein the threads of first and second wire reel parts are all formed in a right-hand thread direction.

FIG. 2 is a diagram illustrating that the threads of a first wire reel part according to an embodiment are formed in the right-hand thread direction, and the threads of a second wire reel part are formed in a left-hand thread direction.

FIG. 3 is a diagram illustrating the first and second wire reel parts, a shaft, a preload-adjusting part, and a motor according to an embodiment.

FIG. 4 is a diagram illustrating the first and second wire reel parts, the shaft, the preload-adjusting part, and the motor according to an embodiment.

FIG. 5 is a diagram illustrating a wire tension adjustment device for a surgical instrument according to a prior art.

FIG. 6 is a diagram illustrating a wire tension compensatory adjustment device according to an embodiment.

FIG. 7 is a block diagram schematically illustrating a wire tension compensatory adjustment device according to an embodiment.

FIG. 8 is a block diagram schematically illustrating a wire tension compensatory adjustment device according to an embodiment.

FIG. 9 is a diagram schematically illustrating a wire tension compensatory adjustment device according to an embodiment.

FIG. 10 is a diagram illustrating a wire tension compensatory adjustment device according to an embodiment.

FIG. 11 is a block diagram schematically illustrating a wire tension compensatory adjustment device according to an embodiment.

FIG. 12 is a block diagram schematically illustrating a wire tension compensatory adjustment device according to an embodiment.

FIG. 13 is a block diagram schematically illustrating a wire tension compensatory adjustment device according to an embodiment.

FIG. 14 is a schematic plan view illustrating a preload applying part according to an embodiment.

FIG. 15 is a schematic plan view illustrating a preload applying part according to an embodiment.

### BEST MODE

Hereinafter, a preferred embodiment of the present disclosure will be described with reference to the drawings. In addition, an embodiment described below does not unduly limit the content of the present disclosure described in the patent claims, and it cannot be said that the entire configuration described in this embodiment is essential as a means of resolving the present disclosure. In addition, description of prior arts and matters that are obvious to those skilled in the art may be omitted, and the omitted descriptions of components (methods) and functions may be sufficiently referenced within the scope of the technical idea of the present disclosure.

A wire tension compensatory adjustment device 10 according to an embodiment is a device that compensates for the elongation of a wire by applying a preload of a long stroke to each of first and second wire reel parts 210 and 220 by using a preload applying part. Hereinafter, the wire tension compensatory adjustment device according to the embodiment will be described in detail with reference to the attached drawings.

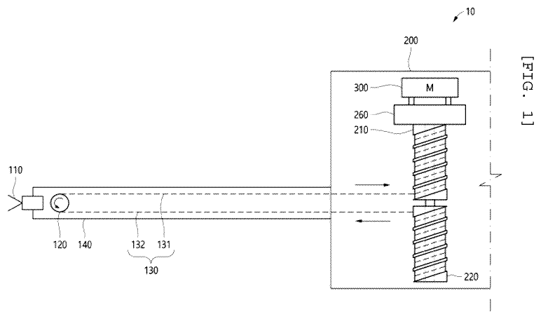

As illustrated in FIG. 1, a wire tension compensatory adjustment device 10 according to the embodiment may include a surgical instrument 110, a pulley part 120, a driving wire 130, and a tube 140.

The surgical instrument 110 according to an embodiment as an end effector is inserted through a lumen provided inside the tube 140 and operates according to a motor provided in a control module part 200 and the traction of the driving wire. In this case, the driving wire is a separate component from the driving wire that pulls the tube. The surgical instrument 110 may have a tube-shaped structure. The surgical instrument 110 and the driving wire controlling the surgical instrument 110 may include a plurality of surgical instruments and a plurality of driving wires, respectively. Preload-adjusting parts may be connected respectively to the driving wires that control the surgical instrument 110. Each of the preload-adjusting parts may maintain the tension of the driving wire. For example, when the driving wire is stretched by a load, the preload-adjusting part may maintain the driving wire to be taut by winding one end of the driving wire. The preload-adjusting part may be provided to correspond to the number of the driving wires.

The pulley part 120 according to an embodiment is disposed on the side of the surgical instrument 110 and rotates so that the driving wire 130 is pulled and released according to the rotation of each of the first and second wire reel parts 210 and 220 as illustrated in FIG. 1.

For example, the driving wire 130 according to an embodiment changes the position or direction of the tube 140 by pulling the tube 140. The driving wire 130 is driven by the operation of the motor 300 disposed in the control module part 200.

For convenience of explanation, the driving wire 130 may be divided into a first driving wire 131 and a second driving wire 132. However, the first driving wire 131 and the second driving wire 132 are connected to each other to constitute one driving wire. In other words, the first driving wire 131 and the second driving wire 132 are formed integrally. The first driving wire 131 is wound or unwound by the rotation of a first wire reel part 210, and the second driving wire 132 is wound or unwound by the rotation of a second wire reel part 220.

Although not shown in the drawing, a joint part and a driving wire may be additionally included to enable two-degree-of-freedom movement of the tube 140, if necessary.

The tube 140 according to an embodiment may be a rigid or flexible tube. In the case of the flexible tube, movement with one degree of freedom or two degrees of freedom is possible by the driving wire as described above.

Meanwhile, in the case of the rigid tube, a preload may be applied to the driving wire that pulls the surgical instrument 110, and in the case of the flexible tube, a preload may be applied to the driving wire 130 that pulls the tube 140. Hereinafter, an example of applying a preload to the driving wire 130 that pulls the tube 140 will be described.

As illustrated in FIG. 1, the control module part 200 controls the driving of the driving wire 130 that pulls the tube 140, and the driving wire (not shown) that drives the surgical instrument 110.

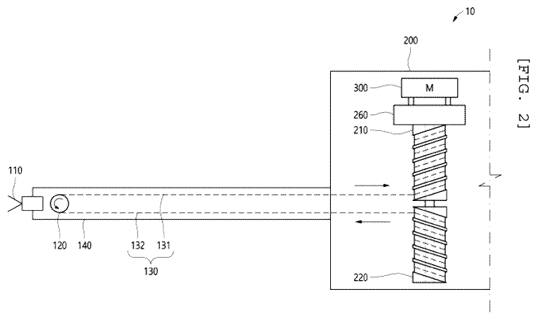

One of the first and second driving wires 131 and 132 illustrated in FIGS. 1 and 2 is wound on the first wire reel part 210, and the other one thereof is wound on the second wire reel part 220. In this case, when one driving wire is unwound according to the control of the motor 300, the other driving wire is wound.

The first wire reel part 210 illustrated in FIG. 1 has threads and grooves formed in a right-hand thread direction, and the first wire reel part 210 illustrated in FIG. 2 has threads and grooves formed in the right-hand thread direction.

Meanwhile, the second wire reel part 220 illustrated in FIG. 1 has threads and grooves formed in the right-hand thread direction, and the second wire reel part 220 illustrated in FIG. 2 has threads and grooves formed in a left-hand thread direction.

On the contrary, although not shown in the drawing, the first wire reel part 210 may have threads and grooves formed in the left-hand thread direction, and the second wire reel part 220 may have threads and grooves formed in the left-hand thread direction or the right-hand thread direction.

The first wire reel part 210 and the second wire reel part 220 rotate in the same directions since rigid bodies thereof are coupled to each other by a shaft 230.

The driving wire 130 controls the position or direction of the tube 140 by winding or unwinding along the formation direction of threads or grooves formed in the first wire reel part 210 and the second wire reel part 220.

Hereinafter, a method of applying a preload through elasticity to the driving wire 130 or the first and second wire reel parts 210 and 220 will be described in detail.

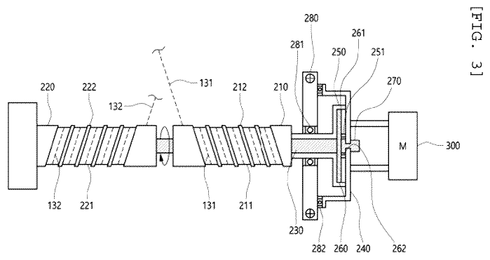

As illustrated in FIG. 3, the rigid bodies of the first and second wire reel parts 210 and 220 are rigidly coupled to each other by the shaft 230. The shaft 230 extends horizontally from the second wire reel part 220 and is coupled to the first wire reel part 210 by extending through the inside of the first wire reel part 210.

The first and second wire reel parts 210 and 220 may rotate in the same directions by the shaft 230. For example, the first wire reel part 210 has grooves 211 and threads 212 formed in the right-hand thread direction. For example, the second wire reel part 220 has grooves 221 and threads 222 formed in the left-hand thread direction.

Each of the first and second wire reel parts 210 and 220 and the preload-adjusting part are supported by a fixing frame 280 so that the first and second wire reel parts 210 and 220 and the preload-adjusting part can rotate according to the operation of the motor 300.

Meanwhile, the first driving wire 131 is wound in a first direction along the direction of the thread formation of the first wire reel part 210 and is fixed at a first fixing point of the first wire reel part 210. The second driving wire 132 is wound in a second direction along the direction of the thread formation of the second wire reel part 220 and is fixed at a second fixing point of the second wire reel part 220.

In this case, based on FIG. 3, the first direction is to the right, and the second direction is to the left. Therefore, the first direction and the second direction are directions moving away from each other, based on the center of the first and second wire reel parts 210 and 220.

The preload-adjusting parts 240, 250, and 260 according to an embodiment may apply a preload to the driving wire 130, thereby preventing slack of the driving wire 130 and maintaining the tension of the driving wire 130. The preload-adjusting part 240, 250, and 260 may operate in a passive manner. The preload-adjusting part 240, 250, and 260 according to an embodiment may include a preload cover 240, a preload base 250, and a preload applying part 260. The preload cover 240 and the preload base 250 may be connected to each other. The preload cover 240 and the preload base 250 may include a space for accommodating the preload applying part 260. The preload base 250 may accommodate the shaft 230. The shaft 230 may pass through the preload base 250. The preload base 250 may support the preload applying part 260.

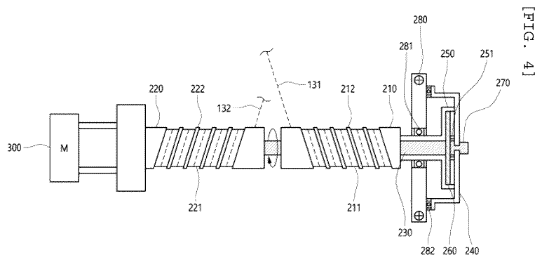

The preload applying part 260 according to an embodiment may be connected to the driving wire 130 to apply a preload to the driving wire 130. The preload applying part 260 may include an elastic body that applies a preload to the first wire reel part or the second wire reel part, and a rotating body that selectively deforms the elastic body. The elastic body may apply a preload to the first wire reel part or the second wire reel part by transmitting power to the first wire reel part or the second wire reel part. For example, the rotating body may be a ratchet wheel or a one-way pulley. It should be noted that the rotating body is not limited to this, and may be replaced with any other component as long as it can perform a similar function. The elastic body may be a spiral spring or a torsional spring. It should be noted that the elastic body is not limited thereto and may be replaced with any other component as long as it can perform a similar function. For example, the preload applying part 260 may include a ratchet wheel and a spiral spring. For example, the preload applying part 260 may include a ratchet wheel and a torsional spring. For example, the preload applying part 260 may include a one-way pulley and a spiral spring. For example, the preload applying part 260 may include a one-way pulley and a torsional spring. For another example, the preload applying part 260 may include only a spiral spring or a torsional spring. In FIGS. 3 and 4, the preload applying part 260 that includes only a spiral spring will be described as an embodiment. A detailed description of various embodiments of the preload applying part 260 will be described later.

The preload applying part 260 according to an embodiment may include a spiral spring. The spiral spring may be seated on the preload base 250 around the axis of the shaft 230. The preload base 250 may be formed by extending from the wire reel part 210. The preload base 250 may have a larger diameter than the wire reel part 210 in order to stably support the preload applying part 260.

The preload cover 240 according to an embodiment may be connected to the preload base 250 and may cover the preload applying part 260. The preload cover 240 may support the shaft 230. The preload cover 240 may be coupled to the preload base 250 in an abutting manner. The shaft 230 may be rotatably connected to the preload cover 240 by a fixing bolt 270.

In this case, in order to apply preload by elasticity to the driving wire 130 or the first and second wire reel parts 210 and 220, the proximal end of the preload applying part 260 is fixed, for example, at a first fixing point 261 on the preload base 250, and the distal end of the preload applying part 260 is fixed, for example, at a second fixing point 262 on one side of a spring fixing part 251.

Meanwhile, the spring fixing part 251 protrudes from the inside of the preload cover 240 toward a spring seating part.

Since the proximal end and the distal end of the preload applying part 260 are fixed at the first and second fixing points 261 and 262, respectively, the preload applying part 260 may be wound or unwound according to the rotation direction of the preload cover 240 (in this case, no rotation of the preload base 250), thereby applying a preload due to elasticity.

When the preload cover 240 is rotated so that the preload applying part 260 has a preset elastic force, the fixing bolt 270 according to an embodiment combines the shaft 230 and the preload cover 240 by bolting. Due to the bolting combination, when the preload cover 240 rotates, the shaft 230 rotates, and in conjunction with this, the first and second wire reel parts 210 and 220 rotate in the same directions.

That is, in the present disclosure, after the bolting combination, the first and second preload-adjusting parts and the first and second wire reel parts 210 and 220 are rotated in the same directions by the shaft 230. Accordingly, when the motor 300 rotates, the first and second preload-adjusting parts, the first and second wire reel parts 210 and 220, and the shaft 230 rotate in the same directions as the operation direction of the motor, and accordingly, the driving wires wound around the first and second wire reel parts 210 and 220 may respectively be wound and unwound or, conversely, may respectively be unwound and wound, depending on the direction of the rotation.

The fixing frame 280 according to an embodiment is fixedly coupled to the body of the control module part 200 to support the shaft 230. In this case, as illustrated in FIG. 3, in order for the preload base 250 to rotate efficiently, a first bearing part 281 is preferably arranged along the circumferential direction of the preload base 250. It should be noted that the first bearing part 281 may be replaced with a bush.

In addition, in order for the preload cover 240 to rotate relative to the fixing frame 280, a second bearing part 282 is preferably disposed between the bottom surface of the preload cover 240 and one surface of the fixing frame 280. It should be noted that the second bearing part 282 may be replaced with a bush.

The motor 300 according to an embodiment is coupled to the preload cover 240, and the preload cover 240 rotates according to the operation of the motor 300.

Meanwhile, as another embodiment of the coupling of the motor 300, as illustrated in FIG. 4, the motor 300 may be disposed on the second wire reel part 220. In the same principle as FIG. 3, when the second wire reel part 220 is rotated by the motor, the first wire reel part 210, the shaft 230, and the first and second preload-adjusting parts 240 and 250 rotate in the same directions.

In addition, although not shown in the drawing, the preload-adjusting part may be disposed on each of the first and second wire reel parts 210 and 220. In this case, the motor 300 may be coupled to any one thereof as illustrated in FIGS. 3 and 4.

When four driving wires are required to drive the tube 140 described above, first and second wire reel parts and preload-adjusting part according to an embodiment may be additionally required. Additional arrangements and configurations may be referenced within the scope of the creation of the technical idea of the present disclosure, in accordance with the principles described above.

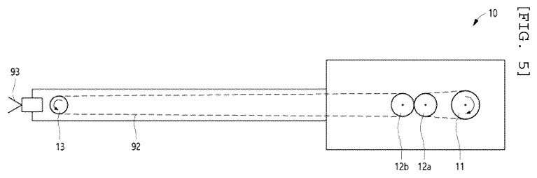

FIG. 6 is a diagram illustrating a wire tension compensatory adjustment device according to an embodiment.

Referring to FIG. 6, a wire tension compensatory adjustment device 20 according to an embodiment may wind or unwind a driving wire 131 and 132, and may apply a preload to the driving wire 131 and 132. The wire tension compensatory adjustment device may include first and second wire reel parts 210 and 220, a shaft 230, a first preload-adjusting part 240, 250, and 260, a first fixing bolt 270, a fixing frame 280, a motor 300, a second preload-adjusting part 440, 450, and 460, a second fixing bolt 470, a second fixing frame 480, and bearing parts 281, 282, 481, and 482. The first wire reel part 210 includes grooves 211 and threads 212. The second wire reel part 220 includes grooves 221 and threads 222.

The first preload-adjusting part 240, 250, and 260 may include a first preload cover 240, a first preload base 250, and a first preload applying part 260. The second preload-adjusting part 440, 450, and 460 may include a second preload cover 440, a second preload base 450, and the second preload applying part 460.

Unless specifically stated, the second preload-adjusting part 440, 450, and 460, the second fixing bolt 470, and the second fixing 480 frame may be components corresponding to the first preload-adjusting part 240, 250, and 260, the first fixing bolt 270, and the fixing frame 280, respectively. For example, the second preload-adjusting part 440, 450, and 460, and the first preload-adjusting part 240, 250, and 260 may be identical to each other.

The wire tension compensatory adjustment device according to an embodiment may be provided with two preload-adjusting parts, and may compensate for the tension of the first driving wire 131 and simultaneously compensate for the tension of the second driving wire 132. The wire tension compensatory adjustment device may adjust the tension of the first driving wire 131 through the first preload-adjusting parts 240, 250, and 260. The wire tension compensatory adjustment device may adjust the tension of the second driving wire 132 through the second preload-adjusting parts 440, 450, and 460.

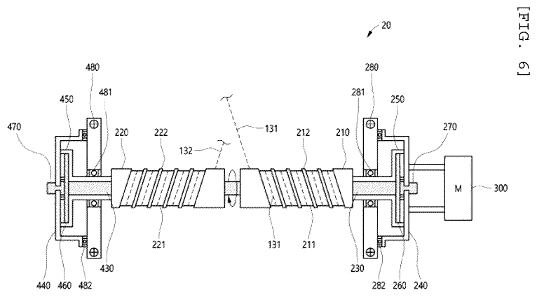

FIG. 7 is a block diagram schematically illustrating a wire tension compensatory adjustment device according to an embodiment.

Referring to FIG. 7, a wire tension compensatory adjustment device 30 according to an embodiment may wind or unwind a driving wire 131 and 132 and may apply a preload to the driving wire 131 and 132. The wire tension compensatory adjustment device may include first and second wire reel parts 210 and 220, a shaft 230, a first preload-adjusting part 260, a second preload-adjusting part 460, and a motor 300. The first wire reel part 210 includes grooves 211 and threads 212. The second wire reel part 220 includes grooves 221 and threads 222. It should be noted that in this specification, for convenience of explanation, the first preload-adjusting part 240, 250, and 260 may be referred to as the first preload-adjusting part 260, or the second preload-adjusting part 440, 450, and 460 may be referred to as the second preload-adjusting part 460.

The first preload-adjusting part 260 and the second preload-adjusting part 460 may be arranged adjacently to each other. For example, based on the longitudinal direction of the shaft 230, the second wire reel part 220, the first wire reel part 210, the first preload-adjusting part 260, the second preload-adjusting part 460, and the motor 300 may be arranged sequentially.

The second preload-adjusting part 460 may apply a preload to the second driving wire 132 by rotating the second wire reel part 220 without interfering with the first preload-adjusting part 260 and the first wire reel part 210. For example, a bearing structure may be provided around the shaft 230 to transmit power from the second preload-adjusting part 460 to the second wire reel part 220.

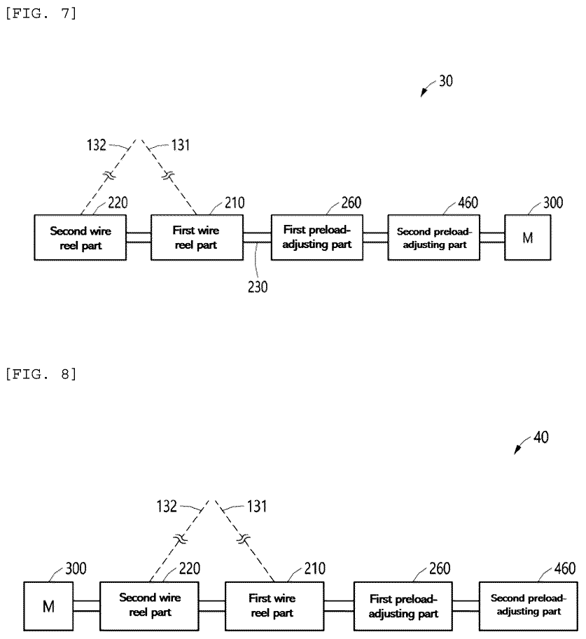

FIG. 8 is a block diagram schematically illustrating a wire tension compensatory adjustment device according to an embodiment.

Referring to FIG. 8, a wire tension compensatory adjustment device 40 according to an embodiment may wind or unwind a driving wire 131 and 132, and may apply a preload to the driving wire 131 and 132. The wire tension compensatory adjustment device may include first and second wire reel parts 210 and 220, a shaft 230, a first preload-adjusting part 260, a second preload-adjusting part 460, and a motor 300. The first wire reel part 210 includes grooves 211 and threads 212. The second wire reel part 220 includes grooves 221 and threads 222. It should be noted that in this specification, for convenience of explanation, the first preload-adjusting part 240, 250, and 260 may be referred to as the first preload-adjusting part 260, or the second preload-adjusting part 440, 450, and 460 may be referred to as the second preload-adjusting part 460.

The first preload-adjusting part 260 and the second preload-adjusting part 460 may be provided adjacently to each other. For example, based on the longitudinal direction of the shaft 230, the motor 300, the second wire reel part 220, the first wire reel part 210, the first preload-adjusting part 260, and the second preload-adjusting part 460 may be arranged sequentially. Based on the first wire reel part 210 and/or the second wire reel part 220, the motor 330 and the preload-adjusting part 260 and 460 may be provided on opposite sides of each other.

The second preload-adjusting part 460 may apply a preload to the second driving wire 132 by rotating the second wire reel part 220 without interfering with the first preload-adjusting part 260 and the first wire reel part 210. For example, a bearing structure may be provided around the shaft 230 to transmit power from the second preload-adjusting part 460 to the second wire reel part 220.

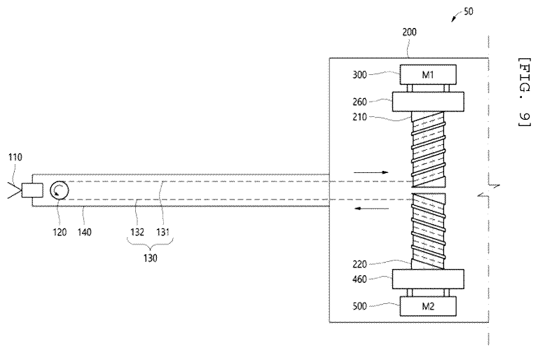

FIG. 9 is a diagram schematically illustrating a wire tension compensatory adjustment device according to an embodiment.

Referring to FIG. 9, a wire tension compensatory adjustment device 50 according to an embodiment may include a surgical instrument 110, a pulley part 120, a driving wire 130, and a tube 140. For example, the tube may be an overtube operated by a driving wire.

The surgical instrument 110 according to an embodiment as an end effector is inserted through a lumen provided inside the tube 140 and operates according to a motor provided in a control module part 200 and the traction of the driving wire. In this case, the driving wire is a separate component from the driving wire that pulls the tube.

The pulley part 120 according to an embodiment is disposed on the side of the surgical instrument 110, and rotates so that the driving wire 130 is pulled and released according to the rotations of the first and second wire reel parts 210 and 220.

As an example, the driving wire 130 according to an embodiment changes the position or direction of the tube 140 by pulling the tube 140. The driving wire 130 operates according to the operation of the motor 300 arranged in the control module part 200.

For convenience of explanation, the driving wire 130 may be divided into the first driving wire 131 and the second driving wire 132. However, the first driving wire 131 and the second driving wire 132 are connected to each other to constitute one driving wire. In other words, the first driving wire 131 and the second driving wire 132 are formed integrally. The first driving wire 131 is wound or unwound by the rotation of a first wire reel part 210, and the second driving wire 132 is wound or unwound by the rotation of a second wire reel part 220.

Although not shown in the drawing, a joint part and a driving wire may be additionally included to enable two-degree-of-freedom movement of the tube 140, if necessary.

The tube 140 according to an embodiment may be a rigid or flexible tube. In the case of the flexible tube, movement with one degree of freedom or two degrees of freedom is possible by the driving wire as described above.

Meanwhile, in the case of the rigid tube, a preload may be applied to the driving wire that pulls the surgical instrument 110, and in the case of the flexible tube, a preload may be applied to the driving wire 130 that pulls the tube 140.

The shaft according to an embodiment may include a plurality of shafts. For example, the shaft may include two shafts. The two shafts may be spaced apart from each other. Among the plurality of shafts, a first shaft may be connected to the first wire reel part 210. Among the plurality of shafts, a second shaft may be connected to the second wire reel part 220. The second shaft is spaced apart from the first shaft. The first wire reel part 210 and the second wire reel part 220 may be spaced apart from each other.

FIG. 10 is a diagram illustrating a wire tension compensatory adjustment device according to an embodiment.

Referring to FIG. 10, a wire tension compensatory adjustment device 60 according to an embodiment may wind or unwind a driving wire 131 and 132, and may apply a preload to the driving wire 131 and 132. The wire tension compensatory adjustment device may include first and second wire reel parts 210 and 220, a shaft 230, a first preload-adjusting part 240, 250, and 260, the first fixing bolt 270, the fixing frame 280, a first motor 300, a second preload-adjusting part 440, 450, and 460, the second fixing bolt 470, the second fixing frame 480, a second motor 500, and a bearing part 281, 282, 481, and 482. The first wire reel part 210 includes the grooves 211 and the threads 212. The second wire reel part 220 includes the grooves 221 and the threads 222.

The first motor 300 may drive the first wire reel part 210, and the second motor 500 may drive the second wire reel part 220. The first preload-adjusting part 260 may be disposed between the first motor 300 and the first wire reel part 210. The second preload-adjusting part 460 may be disposed between the second motor 500 and the second wire reel part 220.

The first preload-adjusting part 240, 250, and 260 may include a first preload cover 240, a first preload base 250, and a first preload applying part 260. The second preload-adjusting part 440, 450, and 460 may include a second preload cover 440, a second preload base 450, and a second preload applying part 460.

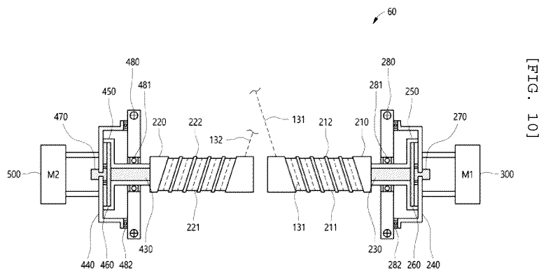

FIG. 11 is a block diagram schematically illustrating a wire tension compensatory adjustment device according to an embodiment.

Referring to FIG. 11, a wire tension compensatory adjustment device 70 according to an embodiment may wind or unwind a driving wire 131 and 132, and may apply a preload to the driving wire 131 and 132. The wire tension compensatory adjustment device may include first and second wire reel parts 210 and 220, a shaft 230, a first preload-adjusting part 260, a second preload-adjusting part 460, a first motor 300, and a second motor 500.

The first wire reel part 210 and the second wire reel part 220 may be physically separated from each other.

The first preload-adjusting part 260 may be disposed between the first motor 300 and the first wire reel part 210. The second preload-adjusting part 460 may be disposed between the second motor 500 and the second wire reel part 220.

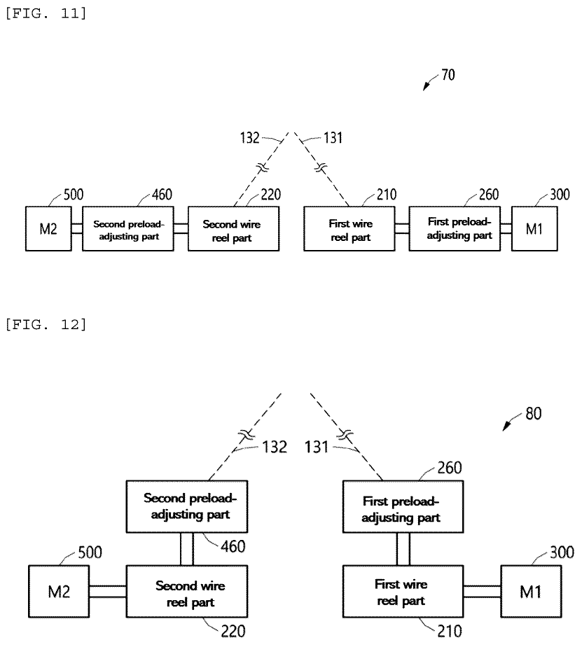

FIG. 12 is a block diagram schematically illustrating a wire tension compensatory adjustment device according to an embodiment.

Referring to FIG. 12, a wire tension compensatory adjustment device 70 according to an embodiment may wind or unwind a driving wire 131 and 132, and may apply a preload to the driving wire 131 and 132. The wire tension compensatory adjustment device may include first and second wire reel parts 210 and 220, a shaft 230, a first preload-adjusting part 260, a second preload-adjusting part 460, a first motor 300, and a second motor 500.

The first wire reel part 210 and the second wire reel part 220 may be physically separated from each other. The first shaft that rotates the first wire reel part 210 may be spaced apart from the second shaft that rotates the second wire reel part 220.

The first driving wire 131 may be wound first on the first preload-adjusting part 260 and then on the first wire reel part 210. The first driving wire 131 may receive a preload from the first preload-adjusting part 260 before being wound on the first wire reel part 210. The position of the first preload-adjusting part 260 is not limited.

The second driving wire 132 may be wound first on the second preload-adjusting part 460 and then on the second wire reel part 220. The second driving wire 132 may receive a preload from the second preload-adjusting part 460 before being wound on the second wire reel part 220. The position of the second preload-adjusting part 460 is not limited.

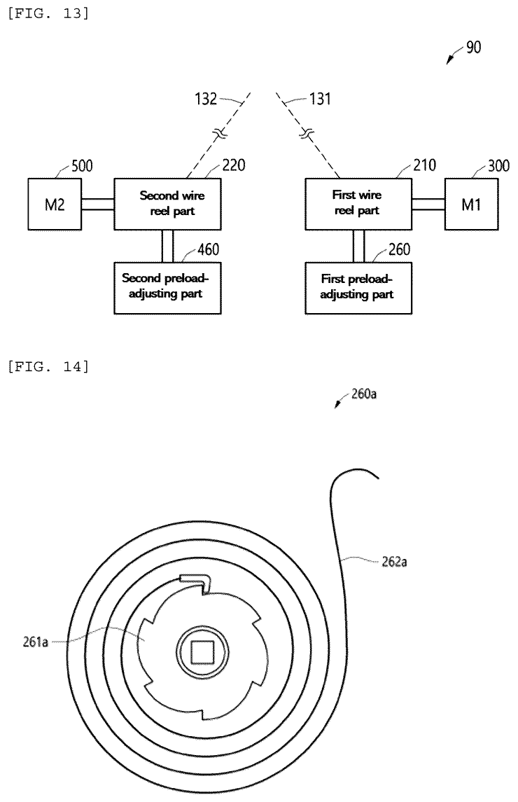

FIG. 13 is a block diagram schematically illustrating a wire tension compensatory adjustment device according to an embodiment.

Referring to FIG. 13, the wire tension compensatory adjustment device 70 according to an embodiment may wind or unwind a driving wire 131 and 132 and may apply a preload to the driving wire 131 and 132. The wire tension compensatory adjustment device may include first and second wire reel parts 210 and 220, a shaft 230, a first preload-adjusting part 260, a second preload-adjusting part 460, a first motor 300, and a second motor 500.

The first wire reel part 210 and the second wire reel part 220 may be physically separated from each other.

The first preload-adjusting part 260 may be located at the rear side of the first wire reel part 210. Here, the rear side refers to a direction opposite to a direction in which the first driving wire 131 extends from the first wire reel part 210. The second preload-adjusting part 460 may be located at the rear side of the second wire reel part 220. The first preload-adjusting part 260 may be connected to the first wire reel part 210 through a wire, or through a power transmission member. For example, the power transmission member may be a band or chain. The second preload-adjusting part 460 may be connected to the second wire reel part 220 through a wire or through a power transmission member. For example, the power transmission member may be a band or chain.

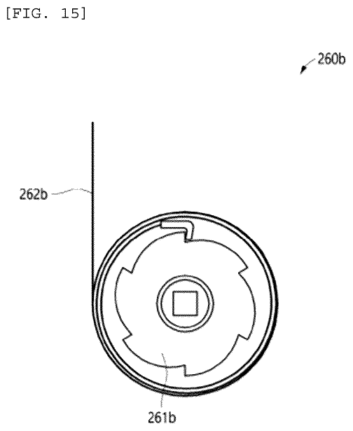

FIG. 14 is a schematic plan view illustrating a preload applying part according to an embodiment.

Referring to FIG. 14, a preload applying part 260a may include a rotating body and a spiral spring 262a. Here, the rotating body may be a ratchet wheel or pulley. In this embodiment, the rotating body is described as a ratchet wheel, but it should be noted that the rotating body is not limited thereto. When the ratchet wheel 261a rotates in the direction of pulling a wire, the ratchet wheel 261a is held by the spring 262a and rotates integrally therewith, so the spring is no longer wound and the force of the spring does not increase further. When the ratchet wheel 261a rotates in the direction of unwinding a wire, the pulley and the motor rotate idly since the ratchet wheel 261a rotates in a direction in which the ratchet wheel 261a is no longer held on a stopper. When the tension of the driving wire is less than the force of the spring, the ratchet wheel 261a is rotated by the spring force, thereby maintaining the tension of the driving wire.

FIG. 15 is a schematic plan view illustrating a preload applying part according to an embodiment.

Referring to FIG. 15, a preload applying part 260b may include a rotating body and a torsional spring 262b. Here, the rotating body may be a ratchet wheel or pulley. In this embodiment, the rotating body is described as a ratchet wheel, but it should be noted that the rotating body is not limited thereto. Regardless of a pulling direction or a releasing direction, the force of the torsional spring 262b always acts uniformly in the pulling direction. However, in the pulling direction, the ratchet wheel 261b is held by a stopper and rotates integrally therewith, so the torsional spring 262b is no longer wound. When the ratchet wheel 261b rotates in the direction of releasing the driving wire, the ratchet wheel 261b and the motor rotate idly since the ratchet wheel 261b rotates in a direction in which the ratchet wheel 261b is no longer held by the stopper. When the tension of the driving wire is less than the force of the spring, the ratchet wheel 261a is rotated by the spring force, thereby maintaining the tension of the driving wire.

In describing the present disclosure, description of prior arts and matters that are obvious to those skilled in the art may be omitted, and the omitted descriptions of components and functions may be sufficiently referenced within the scope of the technical idea of the present disclosure. In addition, the components of the present disclosure described above are explained for the convenience of explanation of the present disclosure, and components not described here may be added as long as they do not deviate from the technical idea of the present disclosure.

The description of the component and function of each part described above is explained separately from each other for convenience of explanation, and if necessary, one component and function thereof may be implemented by integrating with another component, or may be implemented in a more subdivided manner.

Above, the description has been made with reference to an embodiment, but the present disclosure is not limited thereto, and various modifications and applications are possible. That is, those skilled in the art will be able to easily understand that many modifications are possible without departing from the gist of the present disclosure. In addition, it should be noted that when it is determined that detailed description of known functions and components related to the present disclosure, or detailed description of the relationship of the combination of each component of the present disclosure may unnecessarily obscure the gist of the present disclosure, the detailed description thereof is omitted.

1. A wire tension compensatory adjustment device comprising:
a driving wire, wherein the driving wire comprises a first driving wire comprising a first end thereof and a second driving wire comprising a second end thereof, with the first driving wire and the second driving wire being formed integrally;
a first wire reel part capable of winding or unwinding the first driving wire;
a second wire reel part capable of winding or unwinding the second driving wire;
a shaft being connected to the first wire reel part and the second wire reel part to rotate the first wire reel part and the second wire reel part; and
a preload-adjusting part configured to apply a preload to the driving wire to prevent slack of the driving wire and maintain tension of the driving wire.

2. The device of claim 1, wherein the preload-adjusting part operates in a passive manner.

3. The device of claim 1, wherein the preload-adjusting part comprises:
a preload applying part configured to apply a preload to the driving wire by transmitting power to the first wire reel part or the second wire reel part;
a preload base configured to support the preload applying part; and
a preload cover configured to be connected to the preload base and to cover the preload applying part.

4. The device of claim 3, wherein the preload applying part comprises: an elastic body configured to apply a preload to the driving wire; and a rotating body configured to rotate the elastic body.

5. The device of claim 4, wherein the elastic body is a spiral spring or a torsional spring.

6. The device of claim 4, wherein the rotating body is a ratchet wheel or a one-way pulley.

7. The device of claim 1, wherein the preload-adjusting part is provided with a plurality of preload-adjusting parts,
wherein among the plurality of preload-adjusting parts, a first preload-adjusting part is connected to the first wire reel part, and a second preload-adjusting part is connected to the second wire reel part.

8. The device of claim 7, further comprising:
a motor configured to apply power to the shaft to rotate the shaft.

9. The device of claim 8, wherein the motor is provided with a plurality of motors, wherein among the plurality of motors, a first motor drives the first wire reel part, and a second motor drives the second wire reel part.

10. The device of claim 9, wherein the first preload-adjusting part is disposed between the first wire reel part and the first motor, and the second preload-adjusting part is disposed between the second wire reel part and the second motor.

11. The device of claim 9, wherein the shaft is provided with a plurality of shafts,
wherein a first shaft among the plurality of shafts is connected to the first wire reel part,
a second shaft among the plurality of shafts is connected to the second wire reel part and is spaced apart from the first shaft, and
the first wire reel part and the second wire reel part are spaced apart from each other.

12. The device of claim 1, wherein the first driving wire entering the first wire reel part is wound in a first direction, and the second driving wire entering the second wire reel part is wound in a second direction opposite to the first direction.

13. The device of claim 1, wherein when any one wire reel part of the first wire reel part and the second wire reel part has threads and grooves formed therein to wind the driving wire, one remaining wire reel part thereof has threads and grooves formed therein to unwind the driving wire.

14. The device of claim 1, wherein the first wire reel part and the second wire reel part rotate in the same directions when winding or unwinding the driving wire.
